(19) United States
(12) Patent Application Publication
(10) Pub. No.: US 2005/0209626 A1
(43) Pub. Date: Sep. 22, 2005

(54) Needle having vibration-producing handle and method of acupuncture

(75) Inventors: Kyung P. Riihimaki, Weymouth, MA (US); Thomas Riihimaki, Weymouth, MA (US)

Correspondence Address: SUGHRUE MION, PLLC, 2100 PENNSYLVANIA AVENUE, N.W., SUITE 800, WASHINGTON, DC, 20037, US

(73) Assignee: Lhasa OMS, Inc.

(21) Appl. No.: 10/791,378

(22) Filed: Mar. 3, 2004

(51) Int. Cl. A61B 17/34

(52) U.S. Cl. 606/189

(57) ABSTRACT

An acupuncture needle having a handle that causes vibrations in a needle shaft when the handle is rotated, for example, between a practitioner's digits—for example, one or more finger tips and a thumb of a practitioner's hand. The handle includes a specialized cross-sectional shape that causes the vibration during rotation thereof.

### BACKGROUND

An apparatus consistent with the present invention relates to needles and, more particularly, to acupuncture needles. Further, a method consistent with the present invention is generally directed to acupuncture.

### SUMMARY OF THE INVENTION

An illustrative, non-limiting embodiment of the present invention is directed to a needle, for example an acupuncture needle, that vibrates when its handle is rotated. A common acupuncture practice is to twirl the needle during insertion, or during an attempt to grab the Chi or body's energy nerves or centers or acupuncture points. Accordingly, during such practices, or at other times when rotated, a needle consistent with the concepts of the present invention causes a vibration thereby giving an enhanced effect.

### BRIEF DESCRIPTION OF THE DRAWINGS

The aspects of the present invention will become more apparent by describing in detail exemplary, non-limiting, embodiments thereof with reference to the accompanying drawings, wherein like reference numerals designate like or corresponding parts throughout the several views, and wherein:

FIG. 1 is a plan view of an acupuncture needle according to a first embodiment consistent with the concepts of the present invention;

FIG. 2 is a cross-sectional view as taken along line II-II in FIG. 1;

FIG. 3 is an exemplary alternative cross-sectional configuration of a needle handle consistent with the concepts of the present invention;

FIG. 4 is an exemplary alternative cross-sectional configuration of a needle handle consistent with the concepts of the present invention;

FIG. 5 is an exemplary alternative cross-sectional configuration of a needle handle consistent with the concepts of the present invention;

FIG. 6 is an exemplary alternative cross-sectional configuration of a needle handle consistent with the concepts of the present invention;

FIG. 7 is an exemplary alternative cross-sectional configuration of a needle handle consistent with the concepts of the present invention;

FIG. 8 is an exemplary alternative cross-sectional configuration of a needle handle consistent with the concepts of the present invention;

FIG. 9 is an exemplary alternative cross-sectional configuration of a needle handle consistent with the concepts of the present invention;

FIG. 10 is an exemplary alternative cross-sectional configuration of a needle handle consistent with the concepts of the present invention; and

FIG. 11 is a plan view of an acupuncture needle according to a second embodiment consistent with the concepts of the present invention.

### DETAILED DESCRIPTION OF ILLUSTRATIVE, NON-LIMITING EMBODIMENTS CONSISTENT WITH CONCEPTS OF THE PRESENT INVENTION

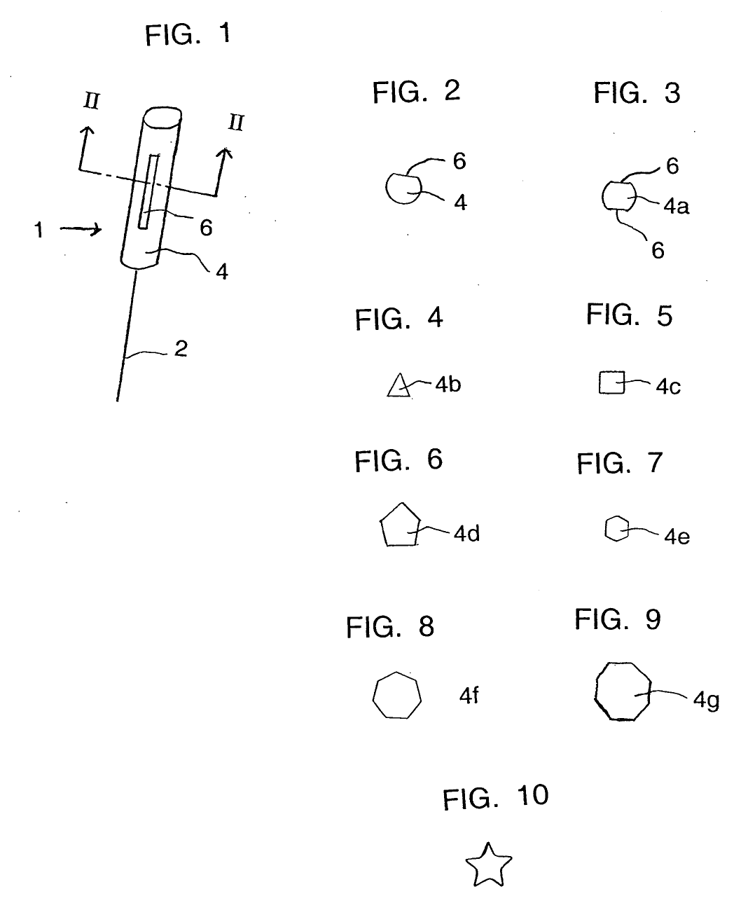

One embodiment, consistent with the concepts of the present invention, is an acupuncture needle 1 as shown in FIG. 1. The acupuncture needle 1 includes a shaft 2 having a tip for insertion into a body. At an end opposite the tip, the shaft 2 includes a body portion that is attached to a handle 4.

The handle 4 includes means for causing a vibration in the shaft 2 when the handle is rotated as, by way of example and not limitation, between a practitioner's digits—for example, one or more finger tips and a thumb of a practitioner's hand. In the present embodiment, the means for causing a vibration includes a flat spot 6 disposed on the handle 4. The flat spot 6 is of such a dimension, in cross-section, as to allow the handle 4 to rotate smoothly and repeatedly. That is, the flat spot 6 is not so large as to cause a stop in rotation as the digits of a practitioner's hand contact it during rotation of the handle 4. The length of the flat spot 6 along a longitude of the handle 4 is not particularly limited, so long as it is big enough to allow vibration when the handle 4 is rotated.

As seen in FIG. 2, a cross-sectional shape of the handle 4 includes an arcuate portion and a chord portion, wherein the chord portion is formed by the flat spot 6. The chord's end points are coincident with those of an arc of less than one-quarter of a circle having a radius corresponding to that of the arcuate portion. By defining the flat spot 6 in such a manner, the handle 4 is allowed to rotate smoothly, yet cause vibration in the shaft 2.

The number of flat spots is not limited to one. Alternatively, as shown in FIG. 3, there may be two flat spots 6. Although the flat spots are shown as being disposed at 180° from one another, they need not be. That is, two flat spots may be disposed adjacent one another, or at any interval. When the flat spots 6 are disposed at other than 180°, the vibration produced thereby will be of an irregular frequency.

Alternatively, three, four, five, six, seven, eight, and so on, flat spots may be disposed around the periphery of the handle 4a, 4b, 4c, 4d, 4e, 4f, and 4g, as respectively shown in FIGS. 4-9. In general, the larger the number of flat spots 6, the higher the frequency of vibration as the handle 4 is rotated. At higher frequencies, the vibration may feel more even and/or continuous and, thus, more pleasing. Although the flat spots 6 are shown as being equiangularly disposed, they need not be, i.e., the handle 4 may have an irregular polygonal shape.

Further, the handle 4 may include a star-shaped cross-section as shown in FIG. 10. Although the star-shaped cross section is shown as being composed of linear segments, the tips of the star may be rounded or, alternatively, the entire “star” shape may be made of alternately disposed concave and convex circular arcs. Still further, although the star-shape is shown as having five arms, such is exemplary, not limiting, so that the star may have any desired number of arms.

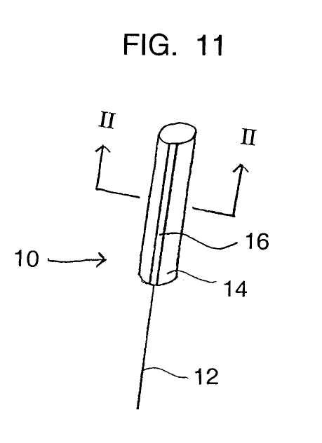

A second embodiment consistent with the concepts of the present invention is shown in FIG. 11. In this embodiment, a needle 10 includes a shaft 12 having a tip at one end thereof, and a handle 14 at the other. The handle 14 is similar to the handle 4 of the first embodiment, except that a flat spot 16 is disposed so as to extend the full length of the handle. In this embodiment, a cross sectional shape of the handle 14 may have any of the shapes as shown in FIGS. 2-10.

The handles 4, 14 may be made from any suitable material such as, by way of example and not limitation, metal or plastic. Also, by way of example and not limitation, the handles 4, 14 may be of a spiral spring type, or pipe type, as is known in the art. Further, the needles 1, 10 may be supplied with or without an insertion guide tube.

Also, the present invention is directed to a method of performing acupuncture including inserting an acupuncture needle into a body of a patient, and rotating a handle of the acupuncture needle so as to cause its shaft to vibrate, thereby causing a pleasing effect to the body.

It is contemplated that numerous modifications may be made to the exemplary embodiments of the present invention without departing from the spirit and scope thereof as defined in the following claims.

1. A needle comprising:
a shaft having a body and a tip; and
a handle attached to said body, said handle including a means for causing a vibration in said shaft when said handle is rotated.

2. The needle as set forth in claim 1, wherein said vibration causing means comprises a portion of said handle having a cross-sectional shape that includes a circular portion and a chord portion.

3. The needle as set forth in claim 1, wherein said vibration causing means comprises a portion of said handle that includes a triangular cross-sectional shape.

4. The needle as set forth in claim 1, wherein said vibration causing means comprises a portion of said handle that includes a rectangular cross-sectional shape.

5. The needle as set forth in claim 1, wherein said vibration causing means comprises a portion of said handle that includes a pentagonal cross-sectional shape.

6. The needle as set forth in claim 1, wherein said vibration causing means comprises a portion of said handle that includes a hexagonal cross-sectional shape.

7. The needle as set forth in claim 1, wherein said vibration causing means comprises a portion of said handle that includes a heptagonal cross-sectional shape.

8. The needle as set forth in claim 1, wherein said vibration causing means comprises a portion of said handle that includes an octagonal cross-sectional shape.

9. The needle as set forth in claim 1, wherein said vibration causing means comprises a portion of said handle that includes a star-shaped cross section.

10. A needle comprising:
a shaft having a body and a tip; and
a handle attached to said body, said handle including a first cross-sectional shape that includes an circular portion and a chord portion, wherein end points of said chord are coincident with those of an arc of less than one-quarter of a circle having a radius corresponding to that of said circular portion.

11. The needle as set forth in claim 10, wherein said handle includes a second cross-sectional shape different from the first cross-sectional shape and disposed at a position spaced longitudinally therefrom.

12. The needle as set forth in claim 10, wherein said first cross-sectional shape includes a second chord portion.

13. The needle as set forth in claim 10 in combination with an insertion guide tube.

14. The needle as set forth in claim 1 in combination with an insertion guide tube.

15. A method of performing acupuncture comprising:
inserting an acupuncture needle into a body of a patient; and
rotating a handle of the acupuncture needle so as to cause its shaft to vibrate.
